(19) United States
(12) Patent Application Publication
(10) Pub. No.: US 2017/0348446 A1
(43) Pub. Date: Dec. 7, 2017

(54) Money Launderer

(71) Applicant: Michael Robert Golden, SR., Lexington, KY (US)

(72) Inventor: Michael Robert Golden, SR., Lexington, KY (US)

(21) Appl. No.: 15/169,742

(22) Filed: Jun. 1, 2016

(51) Int. Cl. A61L 2/10

(52) U.S. Cl. CPC A61L 2/10

(57) ABSTRACT

The present invention is an apparatus that is contained inside the confined space of a Point of Sale Cash Register, Cash Drawer and is designed specifically to sterilize currency and or additional items placed inside the drawer, automatically. The invention includes UVC lighting source(s), either CCFL tube (spanning the length of the enclosure, and located centrally above the denomination separation slots, or by high powered LED-UVC with printed circuit board and located directly above the tray/till slots). The upper, interior surface of the enclosure is lined with highly reflective material. Each slot in the cash tray/till is also lined with highly reflective material. The system is controlled electronically, via an external power supply, that is connected to a dedicated circuit board, which is located in the rear of the enclosure, and is activated by a photo sensor each time the drawer is opened. A programmable timer initializes at the moment the drawer is fully closed. The cycle stops when the drawer is either opened, or the integrated timer expires. A safeguard against undesired leakage from the front of the enclosure, around the drawer opening, is addressed by equipping the cash drawer with a ‘lipped’ door that attaches directly to the horizontally, sliding drawer. The lip wraps around all four sides of the embodiment. UVC is known to be harmful and should be used with necessary precautions in all applications. A counter can be integrated into the circuit to numerically count actual usage over time.

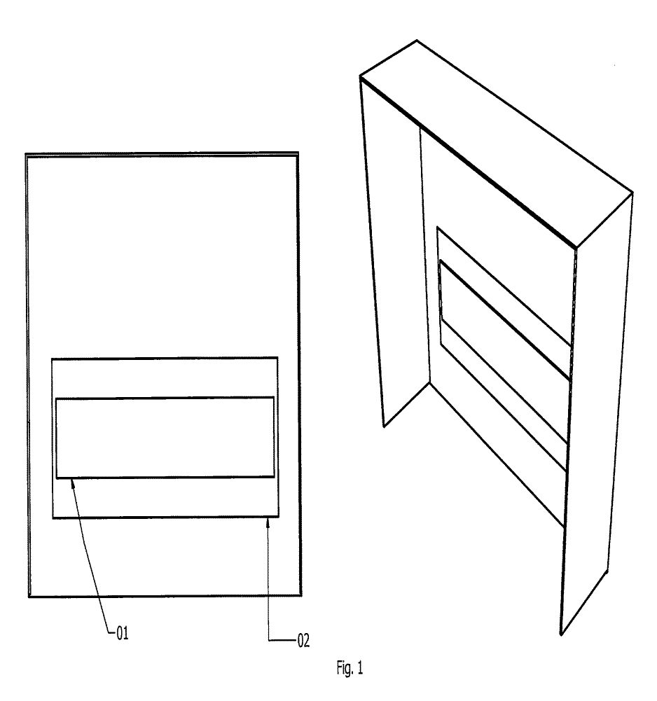

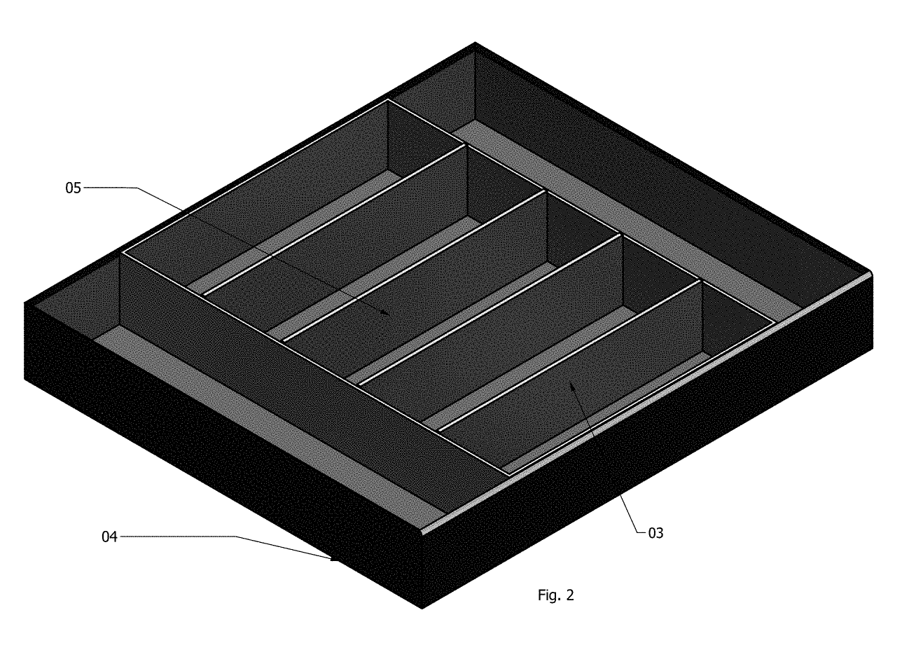

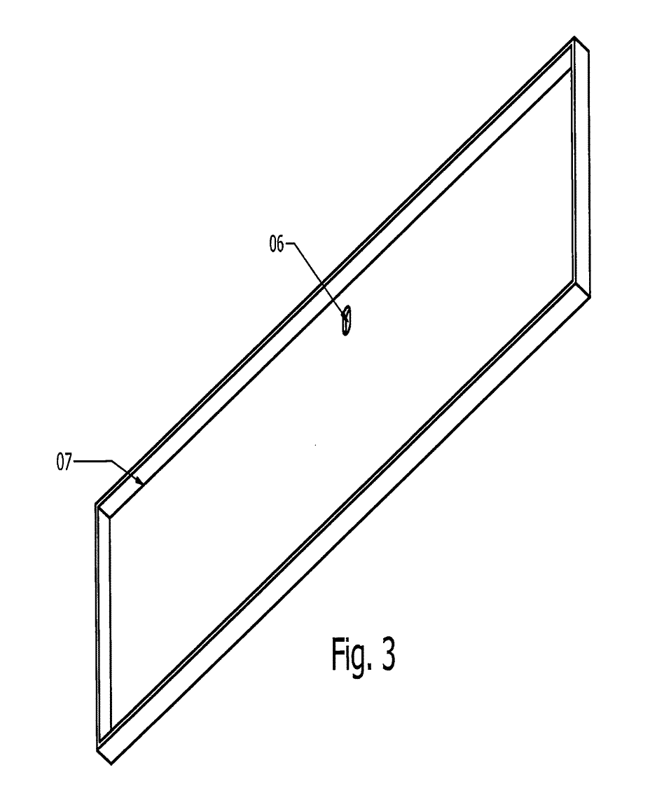

In general, the invention pertains to an apparatus used to sterilize currency inside the confined space of a cash drawer, or point of sale system that is primarily used in all consumer retail markets.

### CROSS REFERENCE

### United States

### Patent Application Publication

### Humphrey's et al.

Pub. No.: US 2004/0099812 A1
Pub. Date May 27, 2004
See also: Application Data Sheet
This application claims benefit to prior filed

### STATEMENT REGARDING FEDERALLY SPONSORED RESEARCH OR DEVELOPMENT

### Not Applicable

### REFERENCE TO SEQUENCE LISTING

### Not Applicable

### BACKGROUND OF THE INVENTION

### 1. Field of the Invention

The present invention generally relates to an apparatus designed for effectively sterilizing microbial and bacterial contaminantion: viruses (e.g., influenza, ebola), bacteria (Ecoli, Shigella, Listeria, Bacillus aureus, Salmonella, etc.) molds and allergens that reside on the currency supply that is in circulation around the world. Specifically, there is not a method for cleaning, disinfecting, sanitizing or sterilizing this circulating cash supply. Because of the materials that cash is composed of, the flu virus, for example, can be active for over 17 days. The flu is responsible for thousands of illnesses each and every year (CDC 2016). Specifically, the invention automatically sterilizes cash inside the cash drawer of retail point of sale systems automatically, each time the drawer is opened and new tender is placed in its respective slot. The invention operates on an automatic cycle that lasts ˜25-30 seconds and then automatically powers of, via a connected, timed relay switch. The invention is capable of destroying active influenza seconds. The apparatus design includes at least one UVC light source (LED, CFL, Mercury Tube, etc.), reflective material (located on the top interior surface of enclosure and lining each individual slot in the sliding cash tray).

### 2. Description of Related Art

The advancement of microbial resistance to antibiotic treatment is increasing (CDC 2015). Each year, the world is faced with an emerging epidemic that creates havoc globally. The cash supply is the vehicle that is responsible for transporting these microscopic killers, globally. Because of the exchange from person to person, everyone is exposed to these threats with no aversion options.

The related arts include those outlined in the device(s) as described by Wesley G. Humphreys. In U.S. Pat. No. 4,786,812 issued on Nov. 22, 1988, Humphreys provides a single piece, hand-held device facilitating the sterilization of a surface contaminated with mold, yeast or virus. Sterilization is achieved via ultraviolet (UV) lamps operating at a wavelength of 253.7 nanometers. The invention further sterilizes the surrounding atmosphere by providing a fan so as to draw an airstream across the UV lamps. Later in U.S. Pat. No. 4,896,042 issued on Jan. 23, 1990, Humphreys provides a two-piece device consisting of a hand-held unit with UV lamps for the sterilization of surfaces and a base unit with fan onto which the hand-held unit is secured for the sterilization of the surrounding atmosphere. This invention is specifically designed to prevent the insertion of objects between hand-held and base units. Neither invention provides for the contained decontamination of high-risk items, namely mail, money, or other commonly encountered objects. Neither invention provides for the neutralization of both chemical and biological threats. Further activity by Humphreys et al. as described in U.S. Pub. No.: US 2004/0099812 provides an embodiment having a slidable disposed tray enabling the insertion of a contaminated object into and the removal of the same object after decontamination from the apparatus. In preferred embodiments, a handle is attached to the slidable tray allowing the user to manually extend and retract the receptacle from the apparatus. In alternate embodiments, the receptacle is mechanically extended and retracted via a motor. A switch is used to activate and deactivate the lamp's based on the position of the tray. Germicidal and thermal lamps are provided to neutralize chemical agents and biological organisms adhered to an object. In alternate embodiments, lamp energy is redirected onto an object via one or more contoured reflectors.

### BRIEF SUMMARY OF THE INVENTION

What is currently required, is a device that allows sterilization: quickly, effectively, automatically and does not require the training of an operator to manage. A system that is economically and environmentally friendly that requires few components. A device that can effectively reduce or eliminate contaminants in a matter of seconds due to the fast pace demands of many retail establishments, such as: gas stations, fast food, restaurants, retail stores, etc. A device that is parameterized by industry standardized dimensions as it pertains to the embodiment of a point of sale system cash drawer, in general, having the dimensions consisting of: 16″ L×16″ W×4″ H. Cash drawers generally have minimal free space inside the enclosure and require specialized lamps, or a complete redesign of the outer casing to accommodate such a system, and to prevent conflicting interference with any internally moving components such as a cash tray/till.

No fans are necessary in this design. All sterilization is conducted via electromagnetic radiation. Each slot will contain reflective material and a magnetic strip that will be used to attract any microorganisms that may be present on the media.

### DETAILED DESCRIPTION OF THE INVENTION

The invention can be installed in existing Point of Sale systems using only components (retro-fit consisting of: UV Germicidal lamp, 5V invertor, relay switch, magnets, reflective material, proximity sensor, power supply, wiring), or manufactured using the same components.

In the retro-fit case, a 4 mm diameter×310 mm long lamp is typically used. In general, a magnetic capacitive proximity sensor is used to detect the drawer, activate the lamp and begin the timed disinfection cycle when the drawer is fully closed.

The manufactured case will typically use LED's (light emitting diodes) that are attached to a printed circuit board and mounted on the top inner surface of the enclosure. The drawer is composed of carbon steel (powder coated). The LED board is connected to another circuit board inside the rear area of the drawer that includes the integrated circuit microchip, timer, photo sensor controller, counter, etc.

The CFL UVC-Germicidal lamp is ˜5 mm in diameter and ˜310 mm long and are composed of quartz glass. They range between 3˜5 watts each. Each lamp is positioned directly above the center of the trays', cash slots. The lamps have two lead wires on either end that are connected to a 5V-high voltage DC invertor. The invertor is connected to the proximity sensor and relay. The sensor is generally placed in a position where it can detect the rear of the cash tray as it locks into position after being closed (on the side of most models by drilling a hole in the exterior casing and mounting it via two nuts). All electronics are powered from a 12V AC/DC power adapter that plugs into a standard 120V AC outlet. The system is integrated into existing POS systems with little to no modifications (depending on the model).

Finally, a new clip system is used to replace old style, spring clips. A magnetic clip system (ferromagnetic clip) is used in each slot to effectively attract any active microorganisms that may be present on the cash. Additionally, due to known penetration issues at the 180-280 nm wavelength, the magnet(s) are used to attract the organism to an opening on either side and between the bills, thus increasing the overall efficacy of the system.

In the description from Humphreys, et al. patent, the device is roughly the size of an ATM machine. Additionally, claiming the use of multiple UV lights (lamps) and also thermal lamps. There is a fan that must be present for the system to perform adequately.

In this invention, reflective material (in general mylar coating, mirrored aluminum, etc.) is on the top of the inner shell of the drawer and lines each individual slot in the sliding tray/till for maximum coverage and effectiveness. The sliding drawer has a face that completely wraps around the opening of the outer casing to prevent leakage, whereby protecting the operator from any exposure to UV radiation. An exterior LED is mounted on the front of the drawer to indicate lamp operation/lamp replacement. A UVC warning label is placed on the front of the drawers' exterior face, between the LED indicator lamp and the manual key/lock. The system has a safety switch located inside the enclosure towards the rear (slotted 2-way beam switch). If the tray is removed, the light will not function.

1. Effective disinfection of currency inside cash drawer
Magnetic tape is positioned between each divider in the cash tray/till to attract microorganisms to the outside edges of the effected media
Reflective material lines the upper surface of the embodiment and antimicrobial/reflective material covers each slot in the sliding tray

2. Space limitations of a standard cash drawer
custom designed high intensity UVC Germicidal lamp source 4 mm diameter×300 mm length (space limitations inside cash drawers)

3. Safe Door with extrusion that surrounds the exterior shell to prevent accidental UV overexposure and or leakage.
Door panel lip
LED that illuminates when the system is in operation
